(19) United States
(12) Patent Application Publication
(10) Pub. No.: US 2004/0073169 A1
(43) Pub. Date: Apr. 15, 2004

(54) Constant pressure apparatus for the administration of fluids intravenously

(76) Inventors: Shai Amisar, Tel Aviv (IL); Baruch Segal, Hod Hasharon (IL); Ronen Radomski, Haifa (IL)

Correspondence Address: BAKER & BOTTS, 30 ROCKEFELLER PLAZA, NEW YORK, NY, 10112

(21) Appl. No.: 10/400,250

(22) Filed: Mar. 27, 2003

Related U.S. Application Data
(63) Continuation-in-part of application No. PCT/IL01/00897, filed on Sep. 25, 2001.

(30) Foreign Application Priority Data
Sep. 28, 2000 (IL) 138766

(51) Int. Cl. A61M 37/00

(52) U.S. Cl. 604/141

(57) ABSTRACT

A constant-pressure apparatus for the administration of fluids intravenously includes a container for a fluid to be administered, and a liquefied-gas source having a constant gas pressure and arranged in association with the container so as to apply the constant pressure of the liquefied-gas cartridge to the fluid therein, thus forcing the fluid to exit through an outlet of the container at a constant rate.

### FIELD OF THE INVENTION

The present invention relates generally, to the administration of fluids intravenously and particularly, to apparatus for the administration of fluids intravenously, wherein the vapor pressure of a liquefied gas controls the fluid pressure, and to a method thereof.

### BACKGROUND OF THE INVENTION

Intravenous administration of fluids to a patient is generally achieved with a fluid bag that is hung somewhat above the patient, generally, at least above the patient's head. The fluid head that is formed provides the pressure necessary to force the fluid into the patient's vein. However, keeping the fluid bag at a height above the patient's head is often cumbersome and uncomfortable, and hinders the patient's mobility. When the patient walks, the bag is generally hung over a special hanger that should be moved around, for example, on wheels.

Other systems of providing constant pressure are known, but they generally involve the use of an electric pump. Thus the patient's mobility is still impaired by the need to be close to a power source.

### SUMMARY OF THE INVENTION

It is one of the objects of the present invention to provide a constant-pressure apparatus for the administration of fluids intravenously, wherein the vapor pressure of a liquefied gas controls the fluid pressure, and to a method thereof.

Thus, in accordance with the present invention, the constant-pressure apparatus is provided for the administration of fluids intravenously, which includes:

a fluid chamber, which contains the fluid to be administered;

a first conduit, in communication with the fluid chamber, for directing the fluid to an intravenous administration needle;

a liquefied-gas cartridge, containing liquefied gas, in communication with the fluid chamber, for providing a constant pressure source to exert pressure onto the fluid chamber, thus forcing the fluid to exit through the first conduit at a constant rate; and

a pressure communicator for communicating the constant pressure of the liquefied-gas cartridge to the fluid chamber.

Further in accordance with the present invention, the apparatus includes a system for strapping the apparatus to a patient's body, in order to maintain the apparatus at body temperature.

Additionally, in accordance with the present invention, the apparatus includes an auxiliary temperature control system.

Further in accordance with the present invention, the auxiliary temperature control system includes a heater, a temperature sensor, and a control unit which controls the operation of the heater, responsive to readings of the temperature sensor.

Alternatively, the auxiliary temperature control system may include a controlled gaseous expansion means, for cooling the apparatus when the vapor pressure within the liquefied-gas cartridge is above a predetermined value.

Further, in accordance with the present invention, the apparatus can include a flow regulator, located on the first conduit, for selectably restricting the rate of fluid administration.

Additionally, in accordance with the present invention, the flow regulator can be electronically controlled for accurately controlling the rate of fluid administration.

Further in accordance with the present invention, the pressure communicator for communicating the constant pressure of the liquefied-gas cartridge to the fluid chamber includes a piston-cylinder arrangement, having:

a cylinder;

a piston, arranged to move freely within the cylinder;

a proximal cylindrical portion on the down-stroke side of the piston; and

a distal cylindrical portion on the up-stroke side of the piston,

wherein the proximal cylindrical portion forms the fluid chamber, and the distal cylindrical portion is in communication with the liquefied-gas cartridge,

and wherein as the liquefied gas evaporates and flows into the distal portion, it exerts constant pressure on the piston, hence on the fluid chamber.

Additionally, in accordance with the present invention, the pressure communicator includes a nozzle for communicating vapor from the liquefied-gas cartridge to the distal cylindrical portion.

Further in accordance with the present invention, the pressure communicator includes a second conduit for communicating vapor from the liquefied-gas cartridge to the distal cylindrical portion.

Additionally, in accordance with the present invention, the pressure communicator includes a valve arranged on the second conduit, for selectably allowing vapor to pass to the distal cylindrical portion, and selectably preventing vapor from passing to the distal cylindrical portion.

Further in accordance with the present invention, the valve has an orifice having a variable diameter, and the valve is arranged to correct for pressure variations in the liquefied-gas cartridge, by decreasing the orifice diameter, responsive to a pressure rise and increasing the orifice diameter, responsive to a pressure drop in the liquefied-gas cartridge.

Alternatively, the pressure communicator for communicating the constant pressure of the liquefied-gas cartridge to the fluid chamber includes a balloon located inside the fluid chamber, in communication with the liquefied-gas cartridge, wherein as the liquefied gas evaporates and flows into the balloon, it exerts constant pressure on the fluid chamber, from within.

Alternatively, the pressure communicator for communicating the constant pressure of the liquefied-gas cartridge to the fluid chamber includes a sleeve, which surrounds the fluid chamber and is arranged to exert pressure on the fluid chamber, wherein the sleeve is in communication with the liquefied-gas chamber.

Further in accordance with the present invention, the liquefied gas is a mixture of several compounds, selected for a specific vapor pressure.

There is thus also provided, in accordance with the present invention, a method of administering fluids intravenously, at a constant pressure, which includes the steps of:

providing a source of liquefied gas of a desired vapor pressure; and

exerting the vapor pressure on a fluid chamber, arranged for intravenous administration of fluids.

Additionally, in accordance with the present invention, the method includes a step of heating the source of liquefied gas to achieve a desired vapor pressure.

Alternatively, the method includes a step of cooling the source of liquefied gas by expanding a portion of the liquefied gas through a conduit that is wound around the source of liquefied gas, to achieve a desired vapor pressure.

Further in accordance with the present invention, the step of cooling the source of liquefied gas includes a step of expanding a portion of the liquefied gas through a valve that is operable to open above a predetermined pressure and operable to close below a predetermined pressure.

Additionally, in accordance with the present invention, the method includes a step of strapping the source of liquefied gas to a patient's body to achieve a working temperature that can generally be the body temperature.

Preferably, there is also provided a pressure regulator associated with the constant pressure source, for regulating the pressure exerted onto the fluid chamber.

In accordance with a further embodiment of the invention, the constant-pressure apparatus is provided for the administration of fluids intravenously, which includes a flexible bag arranged within a pressurizable sleeve and having an outlet, for containing a fluid to be administered; and a liquefied-gas source having a constant gas pressure and arranged in association with the flexible bag so as to apply the constant pressure of the liquefied-gas cartridge to the bag and thus to the fluid therein, thus forcing the fluid to exit through the outlet at a constant rate.

Additionally in accordance with an embodiment of the invention, the sleeve has an inlet, and the liquefied-gas source is a liquefied-gas cartridge arranged externally of the sleeve and having an outlet connected to the inlet of the sleeve.

Further in accordance with an embodiment of the invention, the liquefied-gas source is a liquefied-gas cartridge arranged within the sleeve, and having an outlet arranged in communication with the bag.

Additionally in accordance with an embodiment of the invention, the pressurizable sleeve is an aerosol can having an aerosol valve, wherein the flexible bag is located within the aerosol can and the outlet thereof is connected with the aerosol valve, so as to facilitate selectable delivery therethrough of the fluid.

Further in accordance with an embodiment of the invention, there is also provided a pressure regulator associated with the outlet of the liquefied-gas source, for regulating the magnitude of the pressure applied to the fluid in the bag.

### BRIEF DESCRIPTION OF THE DRAWINGS

The present invention will be more clearly understood from the accompanying detailed description and drawings, in which same number designations are maintained throughout the figures for similar elements and in which:

FIGS. 1A-1C schematically illustrate constant-pressure apparatus for the administration of fluids intravenously, in accordance with an embodiment of the present invention;

FIGS. 2A and 2B schematically illustrate constant-pressure apparatus for the administration of fluids intravenously, in accordance with a second embodiment of the present invention; FIGS. 3A and 3B schematically illustrate constant-pressure apparatus for the administration of fluids intravenously, in accordance with a third embodiment of the present invention; FIGS. 4A-4C schematically illustrate constant-pressure apparatus for the administration of fluids intravenously, in accordance with a fourth embodiment of the present invention; FIGS. 5A-5D schematically illustrate constant-pressure apparatus for the administration of fluids intravenously, in accordance with a fifth embodiment of the present invention; FIGS. 6A and 6B schematically illustrate constant-pressure apparatus for the administration of fluids intravenously, in accordance with a sixth embodiment of the present invention; FIGS. 7A and 7B schematically illustrate constant-pressure apparatus for the administration of fluids intravenously, in accordance with a seventh embodiment of the present invention; FIG. 8 schematically illustrates constant-pressure apparatus for the administration of fluids intravenously, in accordance with an eighth embodiment of the present invention; FIG. 9 illustrates a schematic view of a constant-pressure apparatus including a pressure regulator for the administration of fluids intravenously, according to an embodiment of the present invention;

FIG. 10 illustrates a schematic view of a constant-pressure apparatus including a pressure regulator for the administration of fluids intravenously from an infusion bag, according to another embodiment of the present invention; and

FIG. 11 illustrates a schematic view of an aerosol bag-in-can.

### DETAILED DESCRIPTION

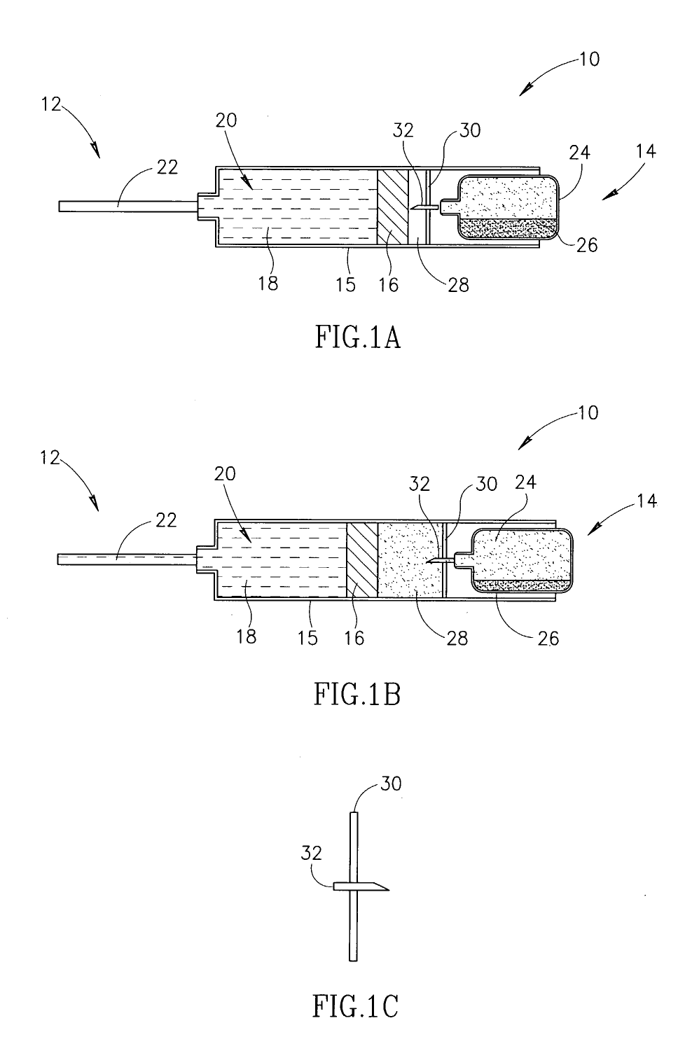

FIGS. 1A-1C schematically illustrate a constant-pressure apparatus 10 for the administration of fluids intravenously, in accordance with a first embodiment of the present invention. Preferably, as shown in FIG. 1A, apparatus 10 is a piston-cylinder like apparatus, which includes a proximal end 12 and a distal end 14, with respect to a patient (not shown). Apparatus 10 further includes a cylinder 15 and a piston 16, arranged to move freely inside cylinder 15. Additionally, apparatus 10 includes a fluid chamber 18, formed as a proximal portion of cylinder 15, on the proximal side of piston 16 and containing a fluid 20 that is to be administered intravenously. Apparatus 10 further includes a fluid conduit 22, for directing fluid 20 to an intravenous administration needle and to the patient. Additionally, apparatus 10 includes a liquefied-gas source, such as a suitable liquefied-gas cartridge 24, containing liquefied gas 26, in communication with a distal portion 28 of cylinder 15, on the distal side of piston 16. Preferably, a sealant 30 seals cylinder 15, so that gas may only enter cylinder 15 via a nozzle 32, such as a hollow needle 32, as shown in FIG. 1C. Nozzle 32 provides communication between liquefied-gas cartridge 24 and distal portion 28 of cylinder 15.

Preferably, as shown in FIG. 1B, as long as liquefied-gas cartridge 24 contains some liquefied gas 26, it is maintained at a vapor pressure which is the specific vapor pressure of liquefied gas 26 at the temperature of apparatus 10, which may be, for example, room temperature. Since liquefied-gas cartridge 24 is in communication with distal portion 28 of cylinder 15, distal portion 28 is also maintained at the specific vapor pressure of liquefied gas 26 at the temperature of apparatus 10. As liquefied gas 26 evaporates and flows into distal portion 28, it exerts pressure on piston 16, pushing it towards proximal end 12, thus providing the administration of fluid 20 to the patient at a constant pressure. Additionally, the constant pressure of fluid administration is the vapor pressure of liquefied gas 26 at the temperature of apparatus 10.

Preferably, a working temperature of apparatus 10 is the ambient temperature of the room. The room may be heated or air-conditioned, and the ambient temperature is generally maintained around 250 C. Alternatively, apparatus 10 may be strapped to the body of the patient and thus maintained at a working temperature, which is generally body temperature of about 370 C.

A specific advantage of the present invention, as illustrated, for example, in FIGS. 1A-1C, is that given the large ratio between the liquid and gaseous states, usually, more than 1:200, a large amount of liquid can be administered intravenously, with a rather compact apparatus.

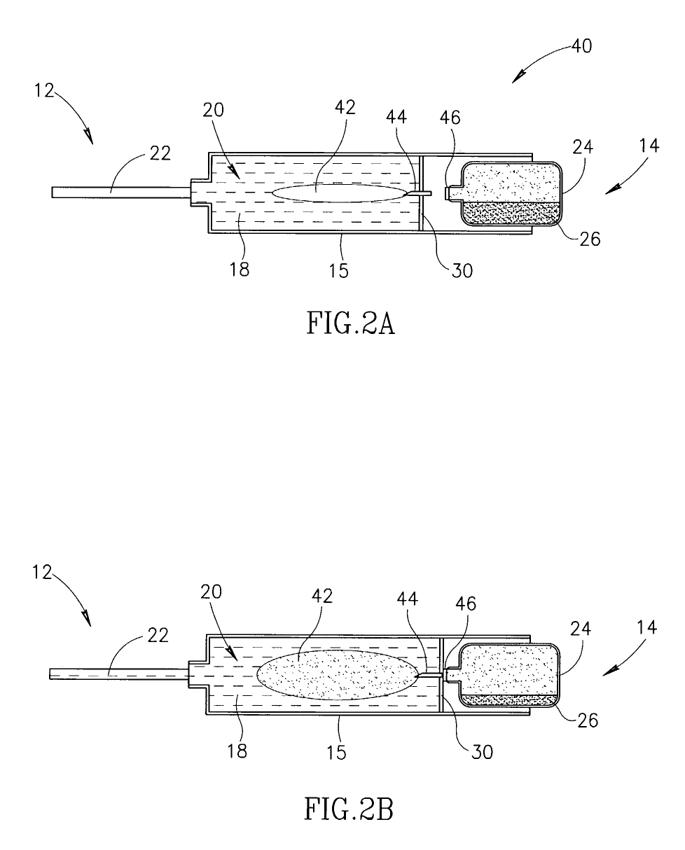

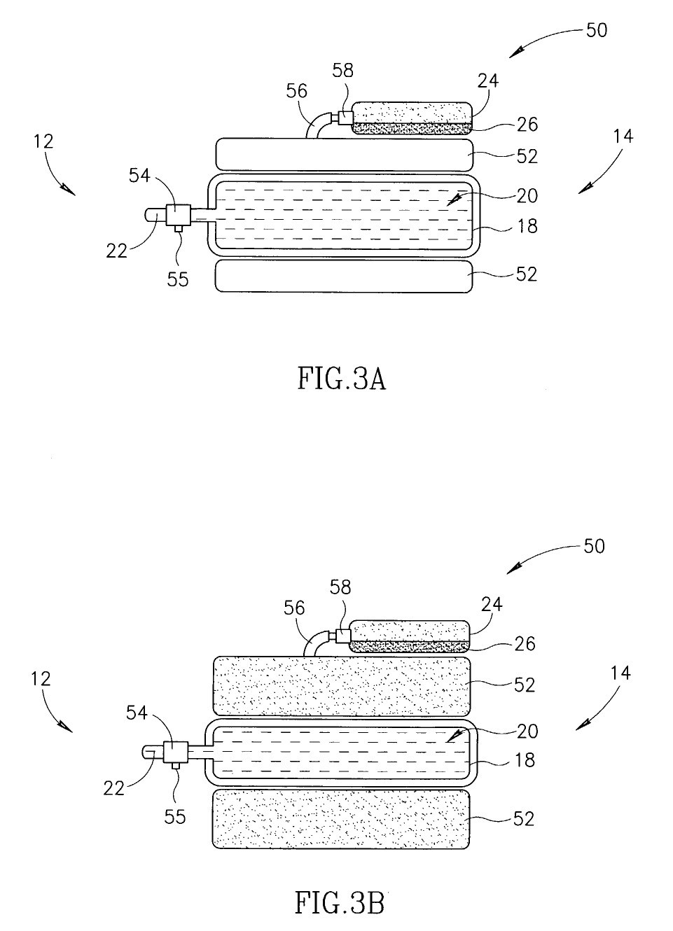

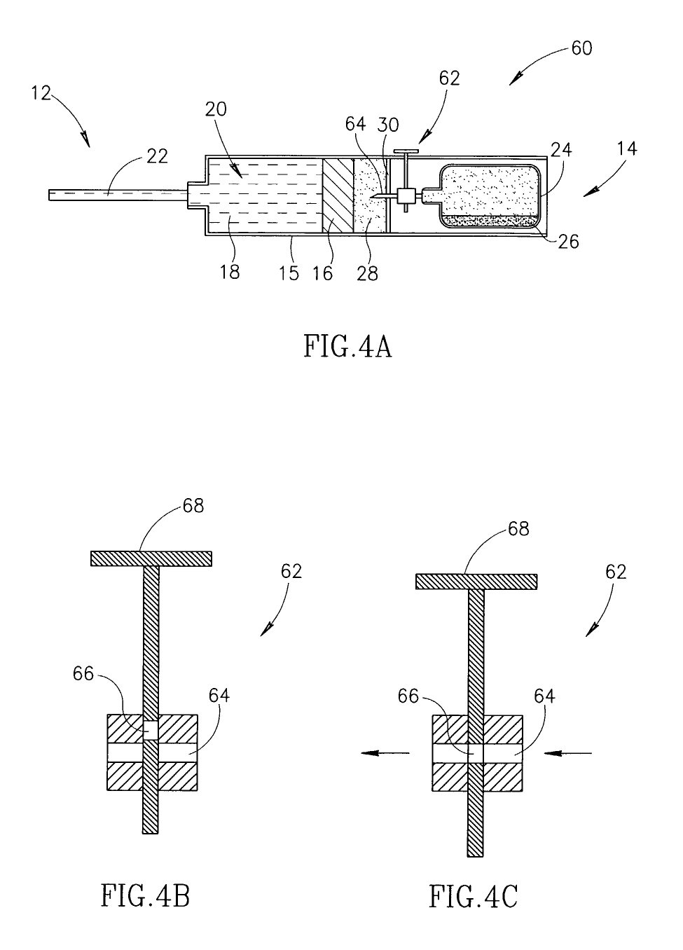

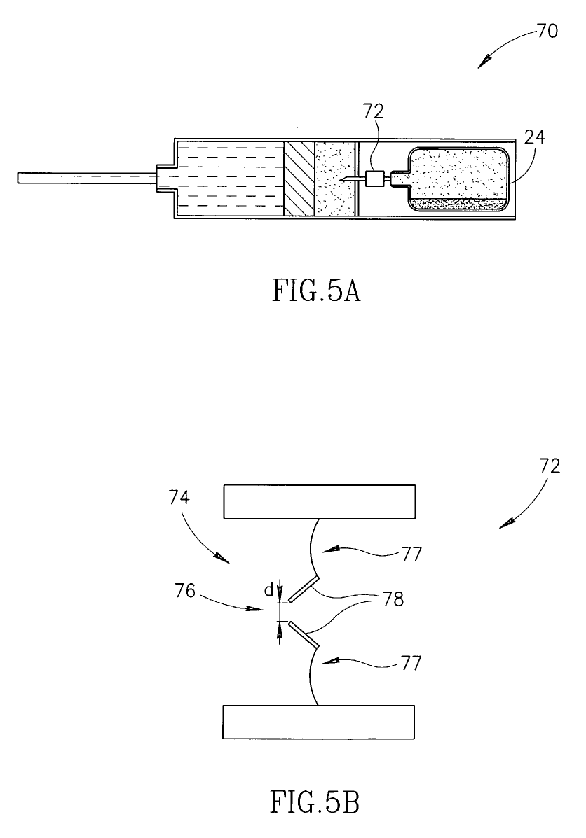

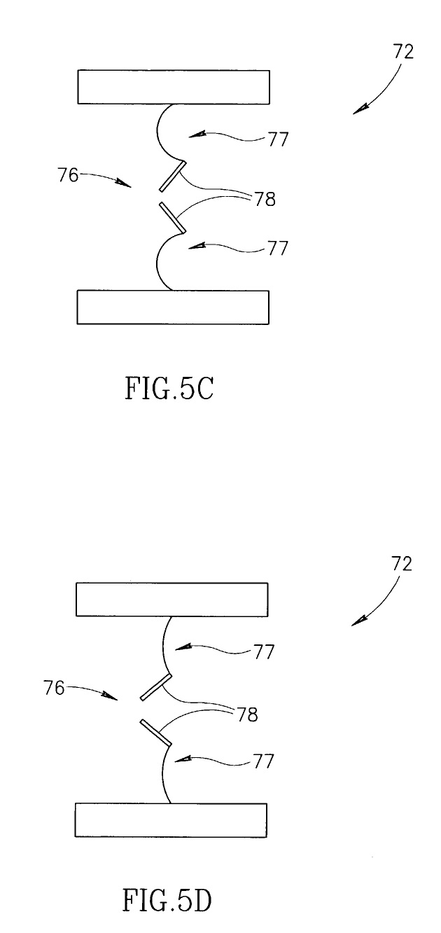

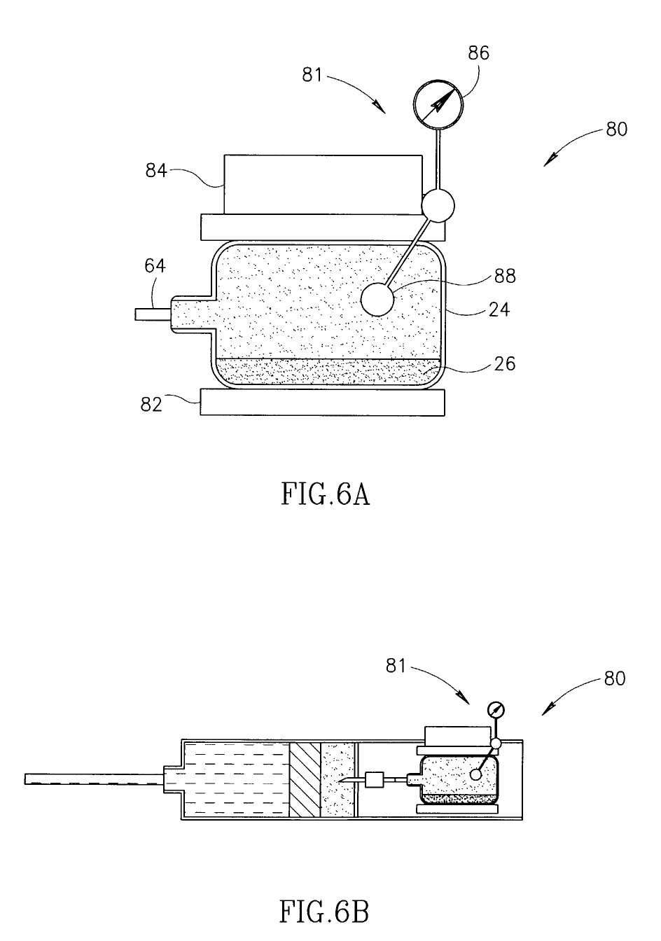

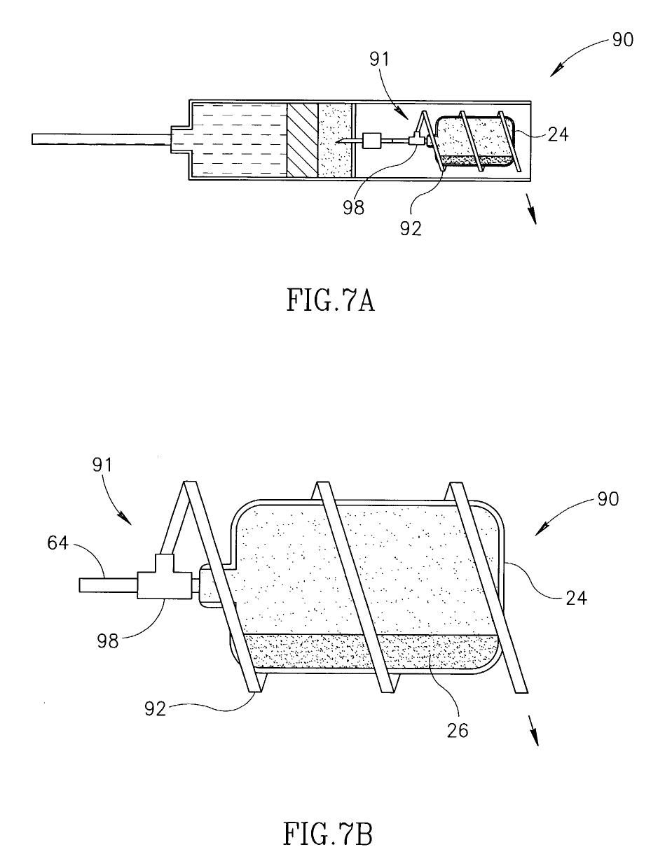

As shown in FIGS. 2A and 2B, which schematically illustrate constant-pressure apparatus 40 for the administration of fluids intravenously, in accordance with a second embodiment of the present invention. Preferably, as shown in FIG. 2A, apparatus 40 includes proximal end 12 and distal end 14, with respect to a patient (not shown). Apparatus 40 further includes cylinder 15, which includes fluid chamber 18, containing fluid 20. Additionally, apparatus 40 includes liquefied-gas cartridge 24, containing liquefied gas 26, in communication with a balloon 42, located inside cylinder 15. The communication between liquefied-gas cartridge 24 and balloon 42 is provided by a hollow needle 44, which is operable to puncture a rubber cap 46 that seals liquefied-gas cartridge 24. Apparatus 40 further includes fluid conduit 22, from which fluid 20 is administered to the patient.

As shown in FIG. 2B, as liquefied gas 26 evaporates and flows into balloon 42, it exerts pressure on fluid chamber 18, pushing fluid 20 out through fluid conduit 22, thus providing the administration of fluid 20 to the patient at a constant pressure. The constant pressure of fluid administration is the vapor pressure of liquefied gas 26 at the temperature of apparatus 40.

Apparatus 40 is generally maintained at room temperature of about 250 C. Alternatively, apparatus 40 may be strapped to the body, and generally maintained at body temperature of about 370 C.

Reference is now made to FIGS. 3A and 3B, which schematically illustrate constant-pressure apparatus 50 for the administration of fluids intravenously, in accordance with a third embodiment of the present invention. Preferably, as shown in FIG. 3A, apparatus 50 includes proximal end 12 and distal end 14, with respect to a patient (not shown). Apparatus 50 further includes fluid chamber 18, containing fluid 20. Additionally, apparatus 50 includes liquefied-gas cartridge 24, containing liquefied gas 26, in communication with a sleeve 52, which surrounds fluid chamber 18. The communication between liquefied-gas cartridge 24 and sleeve 52 is provided by a conduit 56, and preferably also by a valve 58. Apparatus 50 further includes fluid conduit 22, from which fluid 20 is administered to the patient.

As shown in FIG. 3B, as liquefied gas 26 evaporates and flows into sleeve 52, it exerts pressure on fluid chamber 18, pushing fluid 20 out through fluid conduit 22, thus providing the administration of fluid 20 to the patient, at a constant pressure. Additionally, a flow regulator 54 may be provided along fluid conduit 22 to better control or to reduce the fluid administration rate, when desired. Flow regulator 54 may be, for example, a constriction. Alternatively, flow regulator 54 may be a solenoid, operable by a controller 55, when a highly accurate flow rate is preferable, for example, for the administration of a specific medication.

Apparatus 50 is generally maintained at room temperature of about 250 C. Alternatively, apparatus 50 may be strapped to the body, and generally maintained at body temperature of about 370 C.

Reference is now made to FIGS. 4A-4C, which schematically illustrate constant-pressure apparatus 60 for the administration of fluids intravenously, in accordance with a fourth embodiment of the present invention. Apparatus 60 is generally similar in construction to apparatus 10 of FIGS. 1A-1C, but includes a valve 62 and a conduit 64, for providing communication between liquefied-gas cartridge 24 and distal portion 28 of cylinder 15, in place of nozzle 32 (FIGS. 1A-1C).

Preferably, as shown in FIGS. 4B and 4C, valve 62 has a retracted position (FIG. 4B), in which a through channel 66 and conduit 64 are offset with respect to each other, so as not to allow passage of vapor, and a deployed position (FIG. 4C), in which through channel 66 and conduit 64 are aligned with each other, so as to allow passage of vapor. Preferably, valve 62 is manipulated by pushing a handle bar 68 down or pulling handle bar 68 up. In accordance with an alternate embodiment of the present invention, valve 62 is manipulated by rotating handle bar 68 by a 900 turn, to selectably align or misalign through channel 66 with conduit 64.

Reference is now made to FIGS. 5A-5D, which schematically illustrate constant-pressure apparatus 70 for the administration of fluids intravenously, in accordance with a fifth embodiment of the present invention. Apparatus 70 is generally similar in construction to apparatus 10 of FIGS. 1A-1C, but includes a pressure regulator 72, in order to provide better control over the fluid administration rate and to ensure that no sudden surges or drops in administration rate occur.

As shown in FIG. 5B, pressure regulator 72 is formed of a diaphragm 74, having cheek-like portions 77, and beak-like portion 78, with an orifice 76 at the center of beak-like portion 78. Orifice 76 has a diameter d, which decreases in size, as beak-like portion 78 closes, and increases in size, as beak-like portion 78 opens.

As shown in FIG. 5C, when there is a pressure surge in liquefied-gas cartridge 24, for example, as a result of a sudden temperature increase, cheek-like portions 77 inflate, causing beak-like portion 78 to close, constricting orifice 76.

As shown in FIG. 5D, when there is a pressure drop in liquefied-gas cartridge 24, for example, as a result of a sudden temperature drop, cheek-like portions 77 deflate, causing beak-like portion 78 to open, opening orifice 76.

In this manner, pressure regulator 72 provides a generally smooth administration rate.

Reference is now made to FIGS. 6A and 6B, which schematically illustrate constant-pressure apparatus 80 for the administration of fluids intravenously, in accordance with a sixth embodiment of the present invention. Apparatus 80 is generally similar in construction to apparatus 70 of FIG. 5A, but includes an auxiliary temperature control system 81 which includes a heating element 82, preferably powered by a battery 84, for maintaining liquefied-gas cartridge 24 at a constant temperature, preferably above room temperature or body temperature. Preferably, auxiliary temperature control system 81 further includes a control unit 86, such as a thermostat 86 which can be set to a desired temperature, and which is in communication with battery 84. Additionally, auxiliary temperature control system 81 includes a temperature sensor 88, located within liquefied-gas cartridge 24 and in communication with thermostat 86, for controlling the temperature of liquefied-gas cartridge 24. Alternatively, sensor 88 may be located on liquefied-gas cartridge 24.

Together, heater 82, battery 84, thermostat 86 and temperature sensor 88 provide for a system of controlled temperature, hence controlled vapor pressure, for a relatively accurate fluid administration rate.

Reference is now made to FIGS. 7A and 7B, which schematically illustrate constant-pressure apparatus 90 for the administration of fluids intravenously, in accordance with a seventh embodiment of the present invention. Apparatus 90 is generally similar in construction to apparatus 80 of FIGS. 6A and 6B, but includes a controlled gaseous expansion means 91, for cooling apparatus 90 when the vapor pressure within liquefied-gas cartridge 24 is above a predetermined value. Controlled gaseous expansion means 91 includes a valve 98 that is operable to open when the vapor pressure within liquefied-gas cartridge 24 is above a predetermined value. Valve 98 is further operable to close below that predetermined value. Controlled gaseous expansion means 91 further includes a conduit 92 that is wound around liquefied-gas cartridge 24, thus cooling it, when expanded gas flows through it. Consequently, when there is a pressure surge within liquefied-gas cartridge 24, to above the predetermined value, for example, as a result of a temperature increase, valve 98 will open, allowing gas to flow through conduit 92 and to cool liquefied-gas cartridge 24. Cooling will decrease the vapor pressure within liquefied-gas cartridge 24. As the vapor pressure falls below the predetermined value, valve 98 will close.

Reference is now made to FIG. 8, which schematically illustrates a system 100 for strapping constant-pressure apparatus, such as any of apparatus 10, 40, 50, 60, 70, 80, and 90 to the patient's body in order to maintain liquefied-gas cartridge 24 at body temperature. Preferably, system 100 includes a housing 104, preferably formed of a rigid material, which may be easily opened for inserting the apparatus therein, or removing the apparatus therefrom. Additionally, system 100 includes a strap 102 which may be strapped to the patient, for example, around a leg or an arm.

In accordance with the present invention, the different embodiments that have been described provide for different levels of accuracy in the rate of fluid administration. For intravenous feeding, for example, when the level of accuracy in the rate of fluid administration need not be very high, the working temperature of the constant pressure apparatus may be room temperature, which may vary from time to time. As a result, the rate of fluid administration may vary somewhat, with time.

When a better control over the rate of fluid administration is desired, system 100 (FIG. 8) may be used; the constant-pressure apparatus may be strapped to the patient, and generally maintained at body temperature.

When accurate control over the rate of fluid administration is desired, or when it is desired to vary the vapor pressure of a liquefied gas, apparatus 80 of FIG. 6A or apparatus 90 of FIG. 7A may be used.

When a highly accurate rate of fluid administration is desired, for example, for the administration of specific drugs, a solenoid flow regulator 54 (FIGS. 3A and 3B), which may be controlled by controller 55 (FIGS. 3A and 3B) may be used.

Liquefied gas 26 may be, for example, a single compound such as Dymel® A, which is dimethyl ether, CH3OCH3. Alternatively, Dymel® 152a, which is 1,1-diflouroethane CHF2CH3 may be used. Alternatively, butane may be used. Alternatively, liquefied gas 26 may be, for example, a mixture of more than one compound, wherein at the working temperature, the mixture is gaseous. For example, a mixture of Dymel® 134a and Dymel® A or tetrafluoroethane may be used. Additionally, decaflouropentane, ethanol, or water, or the like may be added to the fluid, to lower its gas pressure. Alternatively, other fluids and mixtures of fluids which yield a desired vapor pressure at a desired temperature, may be used.

In general, a vapor pressure of 1.08 bars may be used, at room temperature. Alternatively, other pressures may be used, as needed.

Preferably, a liquefied gas compound or a mixture of more than one compound is selected based on a desired vapor pressure for a specific working temperature.

Referring now to FIG. 9, in accordance with a further embodiment of the present invention, there is illustrated a constant-pressure intravenous infusion apparatus 200. Intravenous fluid is contained in an infusion bag 202 having an outlet 204 formed therefrom. Infusion bag 202 is inserted into a pressurizable sleeve 206, such that outlet 204 extends through an aperture 208, formed in sleeve 206 and thereafter is directed to a patient requiring intravenous infusion.

A liquefied gas source, such as a liquefied gas cartridge 210, is connected in fluid flow communication via a pressure regulator 212, with a gas inlet port 214, formed in sleeve 206, thereby to provide a constant preselected pressure to bag 202.

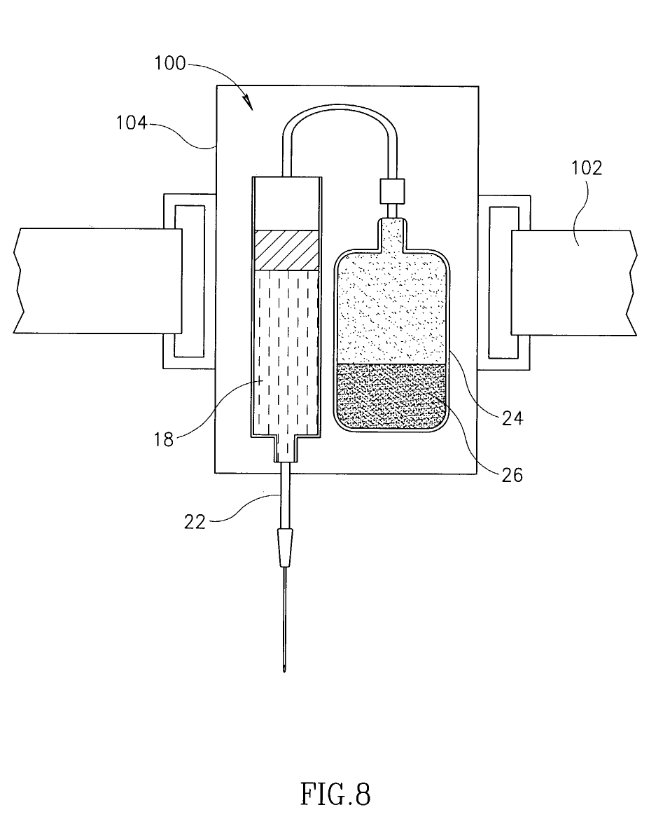

Referring now to FIG. 10, in accordance with another embodiment of the present invention, a constant-pressure intravenous infusion apparatus 200 is provided. Intravenous fluid is contained in an infusion bag 302 having an outlet 304 disposed therefrom. Infusion bag 302 is located within a pressurizable sleeve 306 and intravenous fluid outlet 304 is caused to pass through an aperture 308 formed in the sleeve 306 and thereafter is directed to a patient requiring intravenous infusion.

A liquefied gas source, such as a liquefied gas cartridge 310 is disposed within sleeve 302 and is connected in fluid flow communication via a pressure regulator 312 with a gas inlet port 314 formed in sleeve 306, thereby to provide a constant preselected pressure to intravenous bag 302.

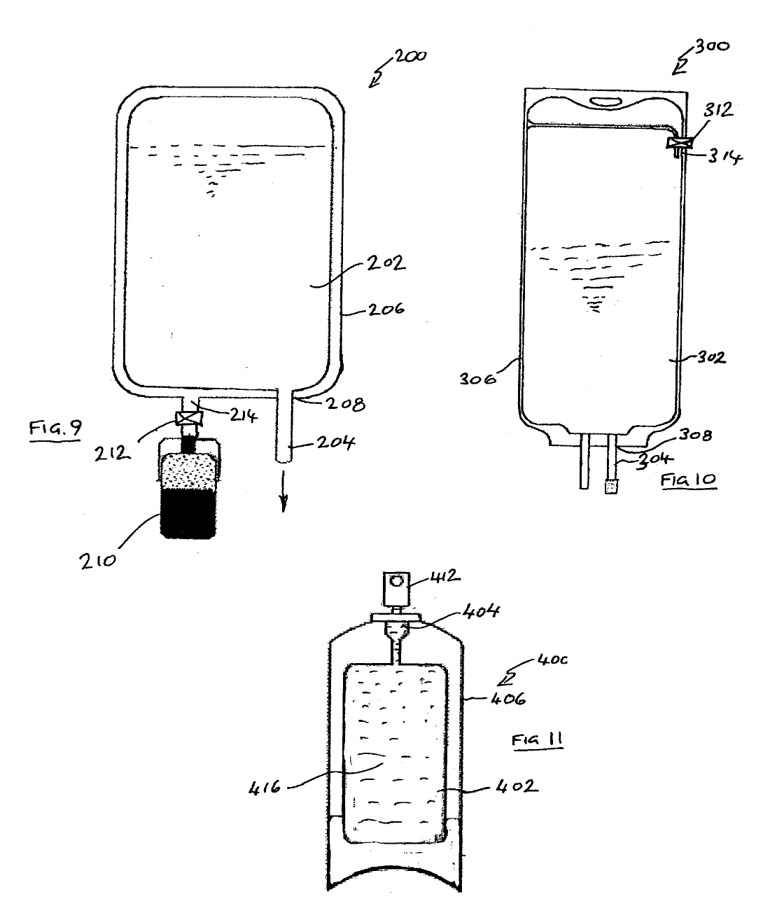

Referring now to FIG. 11, a constant-pressure bag-is illustrated in-can aerosol container 400 for the administration of fluids intravenously according to an alternative embodiment of the present invention. A compressible resilient bag 402 containing intravenous fluid 416 is disposed within aerosol container 406. Bag 402 has an outlet port 404 formed therefrom, connected in fluid flow communication with an aerosol valve 412. Aerosol valve 412 permits the controlled discharge of intravenous fluid 416 through a flow control device (not shown) to a patient requiring intravenous therapy. A so-called piston-in-can aerosol container (not shown) is an alternative to the above-mentioned bag-in-can 400.

It will be appreciated by persons skilled in the art, that the scope of the present invention is not limited by what has been specifically shown and described herein above, merely by way of example. Rather, the scope of the invention is limited solely by the claims, which follow.

What is claimed is:

1. A constant-pressure apparatus for an administration of fluids intravenously, comprising:
a fluid chamber, which contains a fluid to be administered;
a first hose, in communication with said fluid chamber, for directing the fluid to an intravenous administration needle;
a liquefied-gas cartridge, containing liquefied gas, in communication with said fluid chamber, for providing a constant pressure source to exert pressure onto said fluid chamber, thus forcing the fluid to exit through said first hose at a constant rate; and
a pressure communicator for communicating the constant pressure of said liquefied-gas cartridge to said fluid chamber.

2. The apparatus according to claim 1, further comprising a system for strapping the apparatus to a patient's body, in order to maintain the apparatus at body temperature.

3. The apparatus according to claim 1, further comprising an auxiliary temperature control system.

4. The apparatus according to claim 3, wherein said auxiliary temperature control system includes a heater, a temperature sensor, and a control unit which controls the operation of said heater, responsive to readings of said temperature sensor.

5. The apparatus according to claim 3, wherein said auxiliary temperature control system includes a controlled gaseous expansion means, for cooling said apparatus when the vapor pressure within said liquefied-gas cartridge is above a predetermined value.

6. The apparatus according to claim 1, further comprising a flow regulator, located on said first hose, for selectably restricting the rate of fluid administration.

7. The apparatus according to claim 6, wherein said flow regulator is electronically controlled for accurately controlling the rate of fluid administration.

8. The apparatus according to claim 1,
wherein said pressure communicator for communicating the constant pressure of said liquefied-gas cartridge to said fluid chamber includes a piston-cylinder arrangement which has:
a cylinder;
a piston, arranged to move freely within said cylinder, a proximal cylindrical portion on the down-stroke side of the piston; and
a distal cylindrical portion on the up-stroke side of the piston,
wherein said proximal cylindrical portion forms said fluid chamber, and said distal cylindrical portion is in communication with said liquefied-gas cartridge, and
wherein as the liquefied gas evaporates and flows into said distal portion, it exerts constant pressure on said piston, hence on said fluid chamber.

9. The apparatus according to claim 8, further comprising a nozzle for communicating vapor from said liquefied-gas cartridge to said distal cylindrical portion.

10. The apparatus according to claim 8, further comprising a second hose for communicating vapor from said liquefied-gas cartridge to said distal cylindrical portion.

11. The apparatus according to claim 10, further comprising a valve arranged on said second hose, for selectably allowing vapor to pass to said distal cylindrical portion, and selectably preventing vapor from passing to said distal cylindrical portion.

12. The apparatus according to claim 11, wherein said valve has an orifice including a variable diameter, and wherein said valve is arranged to correct for pressure variations in said liquefied-gas cartridge, by decreasing said orifice diameter, responsive to a pressure rise, and by increasing said orifice diameter, responsive to a pressure drop in the liquefied-gas cartridge.

13. The apparatus according to claim 1, wherein said pressure communicator includes a balloon located inside said fluid chamber, in communication with said liquefied-gas cartridge, and wherein, as the liquefied gas evaporates and flows into said balloon, the liquefied gas exerts constant pressure on said fluid chamber, from within said fluid chamber.

14. The apparatus according to claim 1, wherein said pressure communicator includes a sleeve, which surrounds said fluid chamber and is arranged to exert pressure on said fluid chamber, and wherein said sleeve is in communication with said liquefied-gas chamber.

15. The apparatus according to claim 1, wherein said liquefied gas is a mixture of several compounds, selected for a specific vapor pressure.

16. A method of administering fluids intravenously, at a constant pressure, comprising the steps of:
providing a source of liquefied gas of a desired vapor pressure; and
exerting the vapor pressure on a fluid chamber which is arranged for intravenous administration of fluids.

17. The apparatus according to claim 1, further comprising a pressure regulator associated with said constant pressure source, for regulating the pressure exerted onto said fluid chamber.

18. A method of administering fluids intravenously, at a constant pressure, comprising the steps of:
providing a source of liquefied gas of a desired vapor pressure; and
exerting the vapor pressure on a fluid chamber which is arranged for intravenous administration of fluids.

19. The method according to claim 17, further comprising the step of heating the source of liquefied gas to achieve the desired vapor pressure.

20. The method according to claim 17, further comprising the step of cooling the source of liquefied gas by expanding a portion of the liquefied gas through a hose that is wound around the source of liquefied gas, to achieve the desired vapor pressure.

21. The method according to claim 19, wherein said step of cooling the source of liquefied gas further includes a step of expanding a portion of the liquefied gas through a valve that is operable to open above a predetermined pressure, and operable at approximately below a predetermined pressure.

22. The method according to claim 17, further comprising the step of strapping the source of liquefied gas to a patient's body to achieve a temperature that is approximately a body temperature.

23. A constant-pressure apparatus for intravenously administrating fluids, comprising:
a flexible bag arranged within a pressurizable sleeve and having an outlet, said flexible bag adapted for containing a fluid to be administered; and
a liquefied-gas source having a constant gas pressure and arranged in association with said flexible bag so as to apply a constant pressure of said liquefied-gas source to said bag and to the fluid provided therein, and force the fluid to exit through said outlet at a constant rate.

24. The apparatus according to claim 22, wherein said sleeve has an inlet, and said liquefied-gas source is a liquefied-gas cartridge arranged externally from said sleeve and having an outlet connected to said inlet of said sleeve.

25. The apparatus according to claim 22, wherein said liquefied-gas source is a liquefied-gas cartridge arranged within said sleeve, said gas cartridge having an outlet arranged in communication with said bag.

26. The apparatus according to claim 22, wherein said pressurizable sleeve is an aerosol can having an aerosol valve, wherein said flexible bag is located within said aerosol can, and said outlet thereof is connected with said aerosol valve so as to facilitate a selectable delivery therethrough of the fluid.

27. The apparatus according to claim 22, further comprising a pressure regulator associated with said outlet of said liquefied-gas source, for regulating the magnitude of the pressure applied to the fluid in said bag.
